(19) United States
(12) Patent Application Publication
(10) Pub. No.: US 2009/0149962 A1
(43) Pub. Date: Jun. 11, 2009

(54) Artificial Elbow Joint

(75) Inventors: Keiichiro Nishida, Okayama (JP); Hajime Inoue, Okayama (JP); Michihiro Yokokawa, Osaka (JP)

Correspondence Address: WENDEROTH, LIND & PONACK, L.L.P., 1030 15th Street, N.W.,, Suite 400 East, Washington, DC, 20005-1503, US

(73) Assignee: NATIONAL UNIVERSITY CORPORATION OKAYAMA UNIVERSITY, OKAYAMA-SHI OKAYAMA (JP)

(21) Appl. No.: 11/991,556

(22) PCT Filed: Sep. 8, 2006
(86) PCT No.: PCT/JP2006/317848

§ 371 Date: Sep. 29, 2008

(51) Int. Cl. A61F 2/38

(52) U.S. Cl. 623/20.12

(57) ABSTRACT

An artificial elbow joint of constraint type that makes it easier to assemble a joint section even when a surgery for replacing an elbow joint is in process, and is effective in improving the safety of surgery and reducing the time taken to complete the surgery. The artificial elbow joint of the present invention comprises: a humeral component comprising a humeral stem, a shaft and a anterior flange; and an ulnar component comprising an ulnar stem and a sleeve, wherein the shaft of the humeral component is fitted in the sleeve of the ulnar component rotatably. The sleeve has a slit-like sleeve opening a width of which is smaller than an outside diameter of the shaft and having a flexible sleeve insert on inner surface of the sleeve to be contacted with the shaft whereby the sleeve is fitted with a snap into the sleeve through the sleeve opening, and a centerline of the sleeve opening is oriented toward an anterior cubital region at an angle between 45 to 90 degrees with respect to a downward direction of an axis of the ulnar stem.

### BACKGROUND OF THE INVENTION

1. Field of the Invention

The present invention relates to an artificial elbow Joint used in elbow joint replacement surgery, and particularly to an artificial elbow joint of semi-constraint type.

2. Description of the Related Art

Damage on an elbow joint due to rheumatism, osteoarthritis or an injury causes an acute pain as well as the loss of the functions of the elbow joint. In such a case, a surgery to replace the elbow joint with an artificial elbow joint is performed so as to restore the functions of the elbow joint.

The artificial elbow joint comprises a humeral component to be fixed on distal portion of humerus and an ulnar component to be fixed on proximal portion of ulna, of which end portions cooperate to constitute a joint section. The artificial elbow joint is classified roughly into two types by the form of the joint section. One is an artificial elbow joint called the semi-constraint type constituted from a humeral component and an ulnar component that are mechanically connected by means of a hinge mechanism or the like. The other is an artificial elbow joint called the non-constraint type constituted by combining a humeral component and an ulnar component into contact with each other or into releasable fitting relationship when used. In the non-constraint type, the humeral component and the ulnar component are supported by the ligament of an elbow joint. Therefore, use of the artificial elbow joint of non-constraint type for a patient whose ligament of the elbow joint is weakened by aging or other cause may lead to dislocation of the elbow joint. It is considered that an artificial elbow joint of semi-constraint type is more suited to such a patient.

Among the conventional artificial elbow joint of constraint type, such an artificial elbow joint has been known that is disclosed in Japanese Unexamined Patent Publication (Kokai) No. 2000-342610. This artificial elbow joint includes a humerus side member having a rod and a first articular surface, an ulna side member having a rod and a second articular surface and a locking member that is fixed onto the ulna side member by screwing and has a third articular surface. The second articular surface and the third articular surface constitute a continuous articular surface that extends over a range of not less than 180 degrees around the first articular surface and functions as an artificial elbow joint of semi-constraint type.

There is no description on the procedure of the artificial elbow joint replacement surgery in Japanese Unexamined Patent Publication (Kokai) No. 2000-342610, although the configuration of the artificial elbow joint suggests such a procedure as described below. First, the humerus side member and the ulna side member are prepared in a state of not yet assembled, and the rods of these members are inserted into a distal portion of the humerus and a proximal portion of the ulna, respectively. Then the second articular surface is aligned with the first articular surface, and the locking member is fixed onto the ulna side member by screwing so that the third articular surface of the locking member covers the first articular surface, thereby forming the elbow joint.

In the artificial elbow joint replacement surgery commonly practiced, a medullary cavity in which the rods are to be inserted are filled with a bone cement. Then the rods are inserted into the respective bones. When fully hardened, the bone cement achieves the force to fix the artificial elbow joint and the bone together. Accordingly, the positions where the members of the artificial elbow joint are inserted must be determined and forming of the elbow joint must be completed before the cement hardens. However, once the rods have been inserted into the bones, freedom of movement decreases for the humerus side member and the ulna side member, making it difficult to dispose the locking members at proper positions and fix the members in place by screwing. Thus it requires a high skill to dispose the individual components at the optimum positions, assemble the artificial elbow joint and properly position the artificial elbow joint that has been assembled within a limited time before the bone cement hardens.

In addition, to make the joint section function as designed requires it to finish the assembling operation by reliably tightening small screws. However, the surgical practitioner who is forced to complete the complicated operation within the time limit is not capable of eliminating the possibility that the screwing operation would become unreliable. Unreliable screwing may cause the artificial elbow joint to malfunction in a later stage, making a reworking surgery necessary for the elbow joint.

The fact that the burden on the patient increases as the surgery for replacing with the artificial elbow joint takes longer time also strengthens the demand for the reduction of time taken to complete the surgery.

### SUMMARY OF THE INVENTION

An object of the present invention is to provide an artificial elbow joint of semi-constraint type that makes it easier to assemble the joint section during surgery and is effective in improving the safety of surgery and reducing the time taken to complete the surgery.

An artificial elbow joint of the present invention comprises: a humeral component comprising a humeral stem to be inserted in a humerus, a shaft both end of which are fixed on a distal portion of the humeral stem and a anterior flange to hold a cortical bone of the humerus; and an ulnar component comprising an ulnar stem to be inserted in an ulna and a sleeve placed at a proximal portion of the ulnar stem and to receive the shaft, with the shaft of the humeral component being fitted in the sleeve of the ulnar component rotatably. The sleeve has a slit-like sleeve opening a width of which is smaller than an outside diameter of the shaft and having a flexible sleeve insert on inner surface of the sleeve to be contacted with the shaft whereby the sleeve is fitted with a snap into the sleeve through the sleeve opening. A centerline of the sleeve opening is oriented toward an anterior cubital region at an angle between 45 to 90 degrees with respect to a downward direction of an axis of the ulnar stem.

The artificial elbow joint can be assembled by snap-in fitting of the shaft of the humeral component and the sleeve of the ulnar component that constitute the joint section. This feature eliminates the complicated assembling process and screwing operations required in the prior art, so that the elbow joint can be assembled very easily during the surgical operation and the time required in assembling can be reduced. As a result, a sufficient length of time is fixed for assembling the artificial elbow joint by disposing the individual components at the optimum positions and placing the artificial elbow joint in place, thus making the surgical operation easier and safer.

The artificial elbow joint has a specification set forth for the required position where the sleeve opening is to be formed, so that the shaft does not come off the sleeve opening (the so-called dislocation) when a load is applied to the joint section. In the artificial elbow joint of the present invention, the sleeve opening is formed in such a way as the centerline of the opening lies within a range of angles from 45 to 90 degrees from the axis of the ulnar stem directed downward to the anterior cubital region, so that the sleeve opening lies in a direction offset from the vertical direction when bending angle of the elbow joint is within a range from 0 degrees (extended straight) to 90 degrees (bent at right angle). In daily life, one often carries an object with an elbow angle in a range from 0 to 90 degrees. In the artificial elbow joint of the present invention, the shaft does not receive a force in the direction of the sleeve opening when the elbow angle is within this range, and therefore the elbow joint is not likely to experience dislocation in daily life.

In the artificial elbow joint of the present invention, it is preferable that the outer diameter of the shaft and an inner diameter of the sleeve are decreased toward a center in an axial direction thereof.

In the artificial elbow joint of the present invention, it is preferable that the outer diameter of the shaft and the inner diameter of the sleeve are reduced toward the center in the axial direction.

By forming the outer diameter of the shaft and the inner diameter of the sleeve so as to become smaller toward the center in the axial direction, fitting between the shaft and the sleeve can be provided with self-centering function that self-correcting lateral displacement of the joint. With this feature, in case the shaft and the sleeve undergo lateral displacement from a predetermined position relative to each other due to a force applied laterally to the arm, the joint members spontaneously restore the optimum fitting positions when the force is removed. As a result, it is made possible to avoid adverse effects of bending the elbow joint while the joint members are laterally displaced, such as uncomfortable feeling in the joint section or excessive wearing of the members constituting the artificial elbow joint.

In the artificial elbow joint of the present invention, the anterior flange of the humeral component is preferably a separated modular flange having a flange portion and a flange opening having a shape which corresponds to an outer shape of the humeral stem. The modular flange is fixed at the humeral stem by inserting the humeral stem into the flange opening.

Providing the anterior flange as a separate member makes it possible to prepare a plurality of anterior flanges of different sizes and use the anterior flange that best suits the dimensions and shape of the patient's humerus. The conventional anterior flange is integrated with the humeral stem, and the anterior flanges of only one size are available for one-size-fits-all application. This inevitably results in a gap formed between the anterior flange and the humerus which must be filled by bone transplantation before the cement hardens, when an artificial elbow joint is planted. According to the present invention, in contrast, transplantation of bone is eliminated by providing the anterior flange separately so that the anterior flange that is suited to the humerus of the particular patient can be selected.

The artificial elbow joint of the present invention makes it easier to assemble the joint section during surgery and is effective in improving the safety of surgery and reducing the time taken to complete the surgery.

### BRIEF DESCRIPTION OF THE DRAWINGS

FIG. 1 is a side view of an artificial elbow joint according to an embodiment of the present invention.

FIG. 2 is a side view of an ulnar component according to the embodiment.

FIG. 3 is a perspective view of a humeral component according to the embodiment.

FIG. 4 is a perspective view of an anterior flange according to the embodiment.

FIG. 5 is a sectional view of a joint section of the artificial elbow joint according to the embodiment.

### BRIEF DESCRIPTION OF THE REFERENCE NUMERALS

1 Artificial elbow joint
11 Joint section
2 Humeral component
21 Humeral stem
22 Shaft
3 Ulnar component
31 Ulnar stem
32 Sleeve
33 Sleeve insert
34 Sleeve opening
35 Centerline of sleeve opening
36 Axis of ulnar stem
39 Through hole
4 Anterior flange
41 Flange
42 Opening

### DETAILED DESCRIPTION OF THE INVENTION

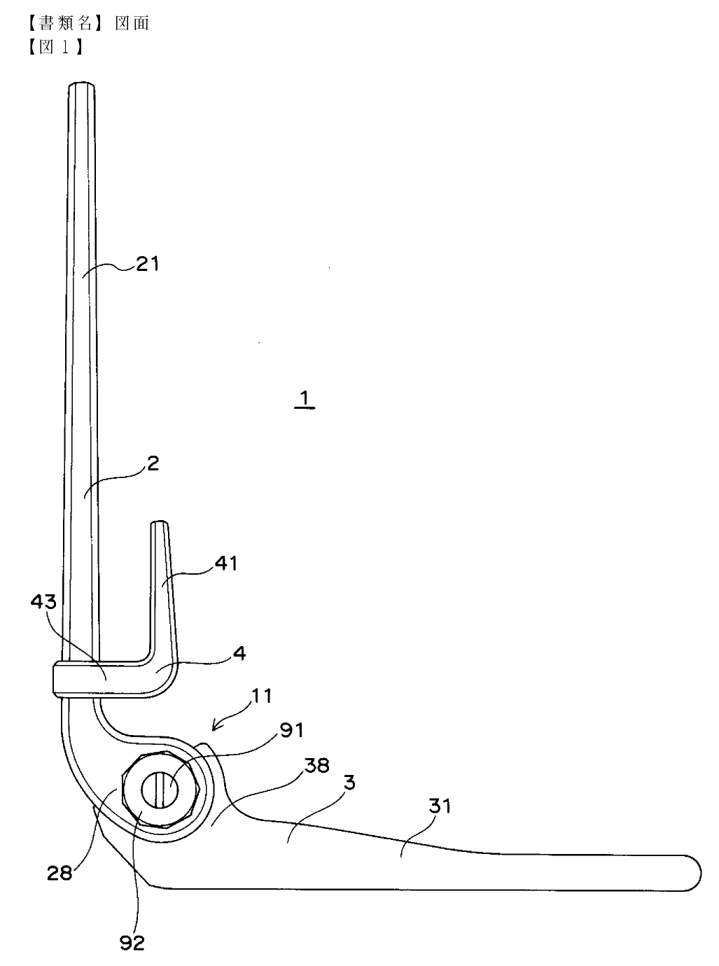

The artificial elbow joint of the present invention is constituted from a humeral component 2 and an ulnar component 3 connected by a joint section 11 as shown in FIG. 1.

FIG. 1 shows the artificial elbow joint bent by about 90 degrees at the elbow joint, with a stem 21 of the humeral component 2 extending vertically and a stem 31 of the ulnar component 3 extending horizontally. The anterior flange 4 is attached to the humeral stem 21 so as to be located within a movable range of the elbow joint.

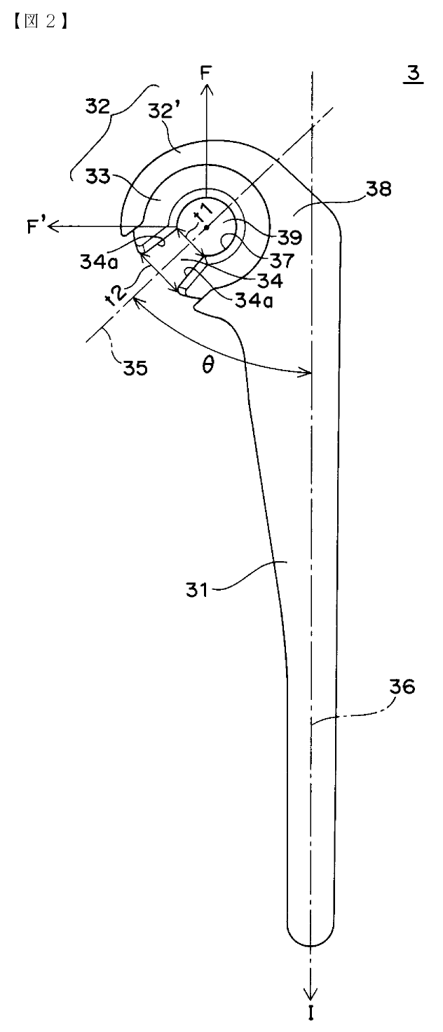

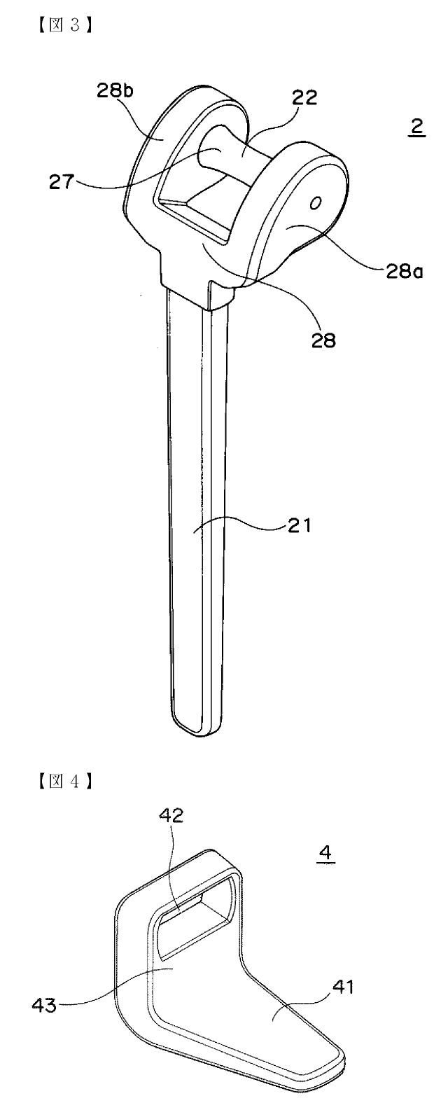

FIG. 2 and FIG. 3 show the ulnar component 3 and the humeral component 2, respectively.

The ulnar component 3 shown in FIG. 2 comprises the ulnar stem 31 to be inserted into a medullary cavity of a proximal portion of ulna and a sleeve 32 formed in a proximal portion 38 of the stem 31. The sleeve 32 comprises a sleeve outer shell 32′ formed from a hard material and a sleeve insert 33 formed from a flexible material.

The sleeve outer shell 32′ has a function mainly to maintain a sufficiently high strength of the sleeve 32, and it is important to support the sleeve insert 33 from the outside so that the sleeve insert 33 that is formed from a flexible material does not deform significantly.

The sleeve outer shell 32′ has an opening that is formed larger than the diameter of the shaft 22 so that the shaft 22 of the humeral component 2 can pass therethrough. However, it is not desirable to make the opening too large which may make it impossible to support the sleeve insert 33. Width and shape of the opening of the sleeve outer shell 32′ are determined so that the sleeve insert 33 can deflect to a proper extent and the sleeve insert 33 does not come off.

The sleeve insert 33 is a portion that receives the shaft 22 of the humeral component 2 to be rotatable therein, and has a through hole 39 having circular cross section for receiving the shaft 22 of the humeral component 2 and a sleeve opening 34 of slit configuration through which the shaft 22 is fitted by snap-in.

The sleeve opening 34 of the sleeve insert 33 is formed so that the centerline thereof lies within a range angles θ from 45 to 90 degrees from the direction I of the axis 36 of the ulnar stem 31 directed downward to the anterior cubital region. When the arm is extended straight downward, direction I of the ulnar component 3 is lies in substantially vertical direction. When carrying a heavy object with hand, the arm is usually extended straight downward, and a reactive force F acts upward in substantially vertical direction in the through hole 39 of the sleeve insert 33. In case the sleeve opening 34 is formed with angle θ in a range from 45 to 90 degrees, there is no possibility of the shaft 22 that is inserted into the sleeve insert 33 being drawn out of the sleeve opening 34 even when the force F is applied.

When the arm is bent to 90 degrees, the direction I becomes horizontal. Relatively light objects may often be carried in this posture, in which case a reactive force F′ perpendicular to the force F acts in the through hole 39 of the sleeve insert 33. In case the sleeve opening 34 is formed with an angle θ of approximately 90 degrees, the force F′ acts in such a direction as the shaft 22 would come off the sleeve opening 34. However, since an object held in this posture is usually not heavy, possibility that dislocation occurs is low.

In order to prevent dislocation. from occurring when the arm is bent, the angle θ is more preferably in a range from 45 to 80 degrees.

Width of the sleeve opening 34 is determined so as to satisfy incompatible demands that it should be easy to insert the shaft 22 and that the shaft 22 should be prevented from coming off. The width of the sleeve opening 34 refers to the width of the opening in the sleeve insert 33. Width of the sleeve opening 34 may be decreased inward. In other words, width t1 on the inside and width t2 on the outside of the sleeve opening 34 may be set to satisfy a relation of t1≦t2, and it is preferable to satisfy a relation of t1<t2. This enables it to make the shaft 22 of the humeral component 2 easy to insert into the sleeve 32 and less likely to come off the sleeve 31.

The sleeve opening 34 can hold the shaft 22 within the sleeve 32 when at least the width t1 on the inside is made smaller than the diameter of the shaft 22.

The width t2 on the outside of the sleeve opening 34 is preferably not larger than the diameter of the shaft 22 for the purpose of preventing the shaft 22 from coming off, but is preferably not smaller than the diameter of the shaft 22 for the purpose of making it easier to insert the shaft 22.

When the widths of the sleeve opening 34 satisfy the relation t1<t2, an inner surface 34a of the sleeve opening 34 inclines so that the width of the sleeve opening 34 becomes smaller toward the inside of the sleeve. While the inner surface 34a is shown in FIG. 2 as being inclined at a constant rate in a straight slope, the present invention is not limited to this configuration and the inclination may be changed. For example, inclination of the inner surface 34 may be changed so that so that the inner surface 34a swells toward the space of the opening 34, that is, the opening 34 is formed in a flaring shape that enlarges toward the outside, which is preferable because it makes it easier to insert the shaft 22 even when t1 is relatively small. The opening 34 of flaring shape also has an effect of making the shaft 22 less likely to come off, since width of the opening 34 decreases abruptly toward the through hole 39 even when t2 is made larger.

The stem 31 and the sleeve outer shell 32′ are formed from a metal of high biocompatibility such as titanium alloy or cobalt-chromium alloy. The sleeve insert 33 is formed from a polymer such as ultra-high molecular weight polyethylene (UHMWPE) that has a low friction coefficient and is less likely to wear. The inner surface of the through hole 39 of the sleeve insert 33 serves as the ulna articular surface 37, and is finished to be very smooth.

The humeral component 2 shown in FIG. 3 comprises the humeral stem 21 to be inserted into the medullary cavity of the distal portion of humerus, a bifurcated distal end portion of humerus 28 formed at the distal end of the stem 21 and the shaft 22 fixed between the two branches of the distal end portion 28. The shaft 22 has a pulley-like shape with the diameter decreasing toward the center. Circumferential surface of the shaft 22 that constitutes the humerus articular surface 27 is formed in a smooth curved surface.

The humeral component 2 comprises the humeral stem 21, the distal end portion of humerus 28 and the shaft 22 that are formed from a metal of high biocompatibility such as titanium alloy or cobalt-chromium alloy.

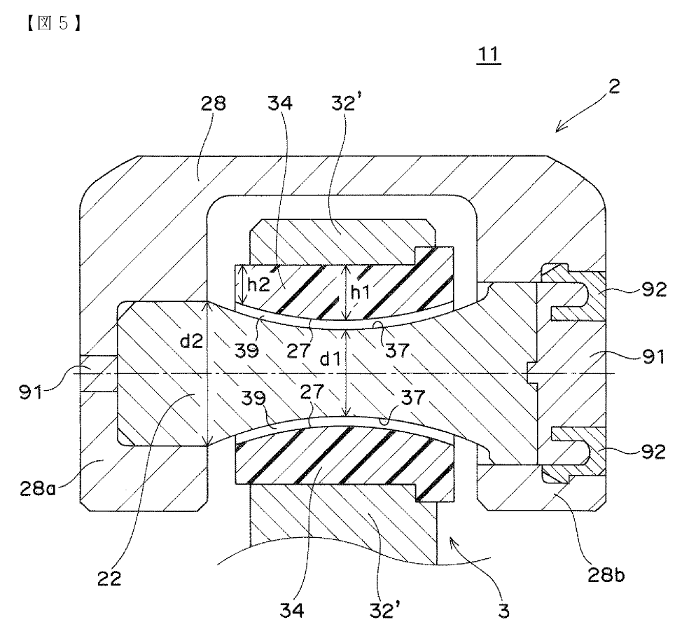

The anterior flange shown in FIG. 4 is of the so-called modular type that is formed separately from the humeral component 2, and will be referred to as modular flange 4 in this specification.

The modular flange 4 is constituted from a flange 41 that holds the cortical bone of the humerus, a flange connection portion 43 formed substantially perpendicular to the flange 42 for connecting the flange 41 to the humeral component 2 and a flange opening 42 formed in the flange connection portion 43 for inserting the shaft 21 therethrough.

The modular flanges 4 with the dimensions and shapes of all parts thereof varied can be prepared. Preparing a plurality of modular flanges with the dimensions of the flange connection portion 43, in particular, is advantageous in that the modular flange 4 that is suited to the thickness of the cortical bone of the patient can be used. The conventional artificial elbow joint is designed with the gap between the humeral stem and the flange that is intentionally set large so as to fit in any person. As a result, when the humeral component 2 is used in a person who has thinned cortical bone, there remains a gap between the flange and the surface of the bone. In such a case, the gap is filled in by transplanting bone in the gap. When the modular flange 4 of the present invention is used, in contrast, the gap between the humeral stem 21 and the flange 4 can be adjusted, thereby providing the artificial elbow joint that enables it to reduce the amount of bone transplantation or makes bone transplantation unnecessary.

FIG. 5 shows the shaft 22, that is fixed at the distal end portion 28 of the humeral component 2, being inserted into the through hole 39 formed in the sleeve insert 34 of the ulnar component 3, thereby constituting the joint section 11 of the artificial elbow joint 1.

The distal end portion 28 of the humeral component 2 is bifurcated, with a recess formed in one distal end portion 28a for fitting the end portion of the shaft 22 therein and an opening formed in the other distal end portion 28b for inserting the shaft 22 therein. The shaft 22 is inserted through the opening of the other distal end portion 28b and, with one end of the shaft 22 fitted in the recess of one distal end portion 28a, the other end of the shaft 22 is fixed in the opening of the distal end portion 28 by means of a pin 91 and a stopper 92.

In the artificial elbow joint 1 of this embodiment, the shaft 22 is formed in a pulley-like shape of which diameter d1 at the center is smaller than the diameter d2 at the ends. The outer surface of the shaft 22 serves as the humerus articular surface 27, and is finished to be very smooth.

As shown in FIG. 5, the sleeve of the ulnar component 3 is constituted by fitting the sleeve insert 34 into the inside of the sleeve outer shell 32. In this example, thickness of the sleeve insert 34 is largest at the center (h1) and decreases toward both ends where the thickness is smallest (h2). Varying the wall thickness in this way results in the inner space of the through hole 39 of the sleeve insert 33 that has a pulley-like shape of which width is smallest at the center, matching the outer shape of the shaft 22. The inner surface of the through hole 39 of the sleeve insert 33 serves as the ulna articular surface 37, and is finished to be very smooth.

Inner diameter of the through hole 39 of the sleeve insert 34 is made slightly larger than the outer diameter of the shaft 22. Thus there is generated a small clearance between the humerus articular surface 27 and the ulna articular surface 37, so as to allow the shaft 22 to rotate smoothly. As a result, the elbow joint can be extended and bent smoothly after the replacement surgery of the artificial elbow joint.

This small clearance gives rise to the possibility of the sleeve insert 34 and the shaft 22 to undergo lateral displacement. However, since the shaft 22 and the sleeve insert 34 of the artificial elbow joint 1 of this embodiment are formed in the pulley-like shape, self-centering effect is achieved so as to restore the original position. As a result, even when a lateral displacement occurs, the lateral displacement can be eliminated by repeating the bending and extending motion of the elbow joint several times.

The self-centering function not only provides the comfort of using the artificial elbow joint but also provides the effect of elongating the service life of the artificial elbow joint. Repeating the bending and extending motions of the elbow joint in the state of the joint section 11 being laterally displaced may cause contacts and/or wear exceeding the level assumed in the design, such as the sleeve 32 making contact with the distal end portion 28 of the humerus or only the mid portion or the ends of the sleeve insert 34 undergoing localized wear. Such a contact or wear which is not taken into account in the design may lead to unexpected dislocation of the elbow joint or shortened service life of the artificial elbow joint.

In case the shaft 22 is formed in a pulley-like shape, it is preferable to form the sleeve opening 34 of the sleeve insert 33 in such a shape that is narrowed at the middle in the front view to match the shape of the shaft, which helps restrict the shaft 22 from coming off.

1. An artificial elbow joint which enables extension and flexion comprising:
a humeral component comprising a humeral stem to be inserted in a humerus, a shaft both end of which are fixed on a distal portion of the humeral stem and a anterior flange to hold a cortical bone of the humerus; and
an ulnar component comprising an ulnar stem to be inserted in an ulna and a sleeve placed at a proximal portion of the ulnar stem and to rotatably receive the shaft,
the sleeve having a sleeve shell and a sleeve insert,
wherein the shaft of the humeral component is fitted in the sleeve of the ulnar component rotatably,
wherein the sleeve has a slit-like sleeve opening a width of which is smaller than an outside diameter of the shaft and having the flexible sleeve insert on inner surface of the sleeve to be contacted with the shaft whereby the sleeve is fitted with a snap into the sleeve through the sleeve opening, and
wherein a centerline of the sleeve opening is oriented toward the rotational direction of the ulnar stem for the case where the elbow is flexed at an angle between 45 to 90 degrees with respect to a vertically-downward direction of an axis of the ulnar stem for the case where the elbow is extended.

2. The artificial elbow joint according to claim 1, wherein the outer diameter of the shaft and an inner diameter of the sleeve are decreased toward a center in an axial direction thereof so that the fitting between the shaft and the sleeve fulfills a self-centering function for self-correcting lateral displacement thereof.

3. The artificial elbow joint according to claim 1, wherein the anterior flange of the humeral component is a separated modular flange having a flange portion and a flange opening having a shape which corresponds to an outer shape of the humeral stem,
wherein the modular flange is fixed at the humeral stem by inserting the humeral stem into the flange opening.

4. The artificial elbow joint according to claim 1, wherein a centerline of the sleeve opening is oriented toward the rotational direction of the ulnar stem when the elbow is flexed at an angle between 45 to 80 degrees with respect to a vertically-downward direction of an axis of the ulnar stem when the elbow is extended.
